(19) United States
(12) Patent Application Publication
(10) Pub. No.: US 2004/0015299 A1
(43) Pub. Date: Jan. 22, 2004

(54) Clustering conformational variants of molecules and methods of use thereof

(75) Inventors: John Hefti, San Francisco, CA (US); Gergely Toth, Palo Alto, CA (US)

Correspondence Address: Charles K. Sholtz, Protein Mechanics, Inc., 280 Hope St., Mountain View, CA, 94041, US

(73) Assignee: Protein Mechanics, Inc., Mountain View, CA (US)

(21) Appl. No.: 10/378,039

(22) Filed: Feb. 27, 2003

Related U.S. Application Data
(60) Provisional application No. 60/359,957, filed on Feb. 27, 2002.

(51) Int. Cl. G06F 19/00; G01N 33/48; G01N 33/50; G01N 31/00

(52) U.S. Cl. 702/19; 702/22

(57) ABSTRACT

Methods of determining and selecting conformational variants of molecules are described. Also disclosed are methods of generating sets of conformational variants, or structures; clustering the conformational variants; and selecting representational variants. In silico analysis methods, such as ligand screening, binding and docking are also disclosed.

### CROSS-REFERENCES TO RELATED APPLICATIONS

This application is entitled to the benefit of the priority filing date of Provisional Patent Application No. 60/359,957, filed Feb. 27, 2002, which is hereby incorporated by reference in its entirety.

### BACKGROUND OF THE INVENTION

The present invention is related to the field of in silico evaluation of molecular structures, and in particular, to the selection of representative molecular structures for use in, e.g., computer-aided screening of putative drugs.

Interactions of molecules of importance to the life sciences, such as proteins and small therapeutic molecules, pose a formidable problem to experimentalists, theorists and computational modelers: experimentally verifying the existence and nature of a given interaction is expensive and time consuming.

One example is in molecular studies involving the binding of potential ligands to protein targets. Pharmaceutical and biotechnology companies are often interested in assessing the binding characteristics of such ligands, which can include lead compounds, NCEs, and/or potential drugs on the targets with which those ligands interact. An accepted system in which to study such interactions is co-crystallization, where putative ligands are crystallized with their protein targets to yield X-ray “co-structures” of target with bound ligand. Such studies are, however, expensive, time consuming, and not suitable for high-throughput studies where one desires to rapidly assess the binding of a multitude of ligands to a particular target molecule.

Experimental limitations such as those mentioned above have given rise to a number of computer-based modeling or simulation approaches to the study of molecular interactions (e.g., the study of ligand-target binding). Here again, however, one runs into limitations. Ideally, a molecular dynamics simulation would be run with each ligand for a sufficiently long period of time to adequately simulate the binding of that ligand to the target molecule. However, due to the long time periods that need to be simulated to get a realistic picture of ligand binding, these approaches are computationally expensive and impractical for high throughput screening applications.

An alternative approach, that addresses the efficiency problems outlined above, is based on computer-implemented fitting of ligands to rigid crystal structure models. This method is much faster than a molecular dynamics approach, since there is no need time component—the fitting is done by minimizing the binding energy of the ligand to the static protein structure. Although this simplification greatly increases the throughput of fitting a number of different ligand structures to the target, it ignores the effects of target-ligand interactions and dynamics of the molecular structure, as well as the obvious steric effects that a rigid model forces on the docking of a ligand. Indeed, rigid structures derived from crystallographic data represent only a very limited subset of all the possible structures which any given target may present to a ligand, and thus will in all likelihood fail to predict many useful interactions.

Attempts have been made to address the limitations of fitting to static crystal structures, e.g., by adding flexibility to the ligand, or incorporating into the protein model some degree of ability to move or flex. However, as these alternative models become more realistic, they invariably become more computationally expensive, such that it is not feasible to use them for rapid screens of thousands or tens of thousands of potential ligand molecules.

Accordingly, it would be desirable to have a computer-based method of modeling molecular interactions (such as target-ligand interactions) that has the efficiency advantage of working with “static” rigid crystal structures, but that also allows takes into considerations the different conformational states that the target may adopt in the process of binding to the ligand. The present invention provides methods which satisfy the above criteria and address the limitations of the prior art.

### SUMMARY OF THE INVENTION

In a general embodiment, the invention provides a method of selecting a plurality of representative structures of a target molecule. The method includes the steps of (i) generating a set of conformational variants of the target molecule, (ii) forming a plurality of clusters of related conformational variants within the set using a clustering algorithm, and (iii) selecting a representative structure from each of the plurality of clusters. The representative structures selected from the clusters together constitute the plurality of representative structures.

In one embodiment, the set of conformational variants is obtained from empirical data, such as a set of NMR structures of the same molecule. In an alternate embodiment, the generating steps includes running a molecular dynamics simulation of the target molecule to generate a series of “snapshots” of the structure, which are then used to form the set of conformational variants.

In one embodiment, the target molecule further includes a ligand binding site. Here, if the conformational variants are generated using a molecular dynamics simulation, the simulation may include, in one embodiment, a simulation which simulates or models an exemplary ligand bound at the ligand binding site. In a related embodiment, the clustering is performed using only residues that are in proximity to the ligand binding site, e.g., as illustrated in Example 1.

Various embodiments of the invention will use various clustering algorithms. For example, in one embodiment, the clustering algorithm is based on partitioning around medoids; in another embodiment, the clustering algorithm uses “fuzzy” clustering; in yet another embodiment, it employs linkage clustering; and in still another embodiment, hierarchical clustering is used.

The invention includes embodiments where the representative structure from at least one of the clusters is a flexible structure, such as a dynamic pharmacophore. In an alternative embodiment, the representative structure from at least one of the clusters is a rigid structure, such as a structure contained in one of clusters. In a specific embodiment, such a representative structure is the one that has the smallest deviation from the average of the structures forming the relevant cluster.

In a broad embodiment, the target molecule is a protein, such as a receptor or any other protein described herein. In such embodiment, the target molecule may be based on a crystal structure, an NMR structure, or on other empirically-determined data. Alternatively, in another embodiment, the target molecule is based on a predicted structure.

In another broad embodiment, the methods of the invention summarized above further include performing an in silico analysis using the representative structure and one or more ligands. The ligands may be, e.g., small molecules, biomolecules (e.g., peptides), biopolymers, endogenous ligands, and the like. In one set of embodiments, the in silico analysis is selected from the group consisting of in silico screening, in silico docking, in silico lead discovery, and in-silico lead optimization. For example, the in silico analysis may include screening a plurality of ligands against the molecule. In specific embodiments, the method includes screening at least ten ligands; in a related embodiment, at least 20 ligands; in another related embodiment, at least 30 ligands; in another related embodiment, at least 50 ligands; in another related embodiment, at least 100 ligands; in another related embodiment, at least 200 ligands; in another related embodiment, at least 300 ligands; in another related embodiment, at least 400 ligands; in another related embodiment, at least 500 ligands; in another related embodiment, at least 1000 ligands; in another related embodiment, at least 10,000 ligands; in another related embodiment, at least 10,000 ligands; and in another related embodiment, at least 100,000 ligands.

Also included as part of the invention is a method of assessing the activity (e.g., binding activity, docking activity, etc) of a plurality of ligands on a target molecule. The method includes the steps of (i) providing a plurality of representative structures obtained by clustering a set of conformational variants of the target molecule, and (ii) using the plurality of representative structures as targets in in silico analysis of the plurality of ligands, where results of the in silico analysis are effective to assess activity of the plurality of ligands on the target molecule. In one embodiment, the set of conformational variants is obtained from empirical data, such as a set of NMR structures of the same molecule. In an alternate embodiment, the set of conformational variants is obtained by running a molecular dynamics simulation of the target molecule to generate a series of “snapshots” of the structure, which are then used to form the set of conformational variants.

In one embodiment, the target molecule further includes a ligand binding site. Here, if the conformational variants are generated using a molecular dynamics simulation, the simulation may include, in one embodiment, a simulation which simulates or models an exemplary ligand bound at the ligand binding site. In a related embodiment, the clustering is performed using only residues that are in proximity to the ligand binding site, e.g., as illustrated in Example 1. Various clustering algorithms may be used. For example, in one embodiment, the clustering algorithm is based on partitioning around medoids; in another embodiment, the clustering algorithm uses “fuzzy” clustering; in yet another embodiment, it employs linkage clustering; and in still another embodiment, hierarchical clustering is used.

The invention includes embodiments where the representative structure from at least one of the clusters is a flexible structure, such as a dynamic pharmacophore. In an alternative embodiment, the representative structure from at least one of the clusters is a rigid structure, such as a structure contained in one of clusters. In a specific embodiment, such a representative structure is the one that has the smallest deviation from the average of the structures forming the relevant cluster.

In a broad embodiment, the target molecule is a protein, such as a receptor or any other protein described herein. In such embodiment, the target molecule may be based on a crystal structure, an NMR structure, or on other empirically-determined data. Alternatively, in another embodiment, the target molecule is based on a predicted structure.

In another broad embodiment, the ligands may be, e.g., small molecules, biomolecules (e.g., peptides), biopolymers, endogenous ligands, and the like. In one set of embodiments, the in silico analysis is selected from the group consisting of in silico screening, in silico docking, in silico lead discovery, and in silico lead optimization. For example, the in silico analysis may include screening a plurality of ligands against the molecule. In specific embodiments, the method includes screening at least ten ligands; in a related embodiment, at least 20 ligands; in another related embodiment, at least 30 ligands; in another related embodiment, at least 50 ligands; in another related embodiment, at least 100 ligands; in another related embodiment, at least 200 ligands; in another related embodiment, at least 300 ligands; in another related embodiment, at least 400 ligands; in another related embodiment, at least 500 ligands; in another related embodiment, at least 1000 ligands; in another related embodiment, at least 10,000 ligands; in another related embodiment, at least 10,000 ligands; and in another related embodiment, at least 100,000 ligands.

In a series of embodiments broadly applicable to any of the above methods, the plurality of representative structures consists of, in one embodiment, at least five representative structures; in another embodiment, at least ten representative structures; in yet another embodiment, at least twenty five representative structures; in still another embodiment, at least fifty representative structures; and in additional embodiments, at least 100, 150, 200, 250, 300, 350, 400, 450, and 500 representative structures.

The present invention also provides for a computer system and computer code. The computer system preferably includes at least one processor and an associated memory subsystem, where the memory subsystem holds computer code to instruct the at least one processor to carry out any of the methods described herein.

It will be appreciated by one of skill in the art that the embodiments summarized above may be used together in any suitable combination to generate additional embodiments not expressly recited above, and that such embodiments are considered to be part of the present invention.

### BRIEF DESCRIPTION OF THE DRAWINGS

FIG. 1 shows an abstraction of the representational space of all possible conformations for a selected molecule, and three exemplary conformations identified in the representational space.

FIG. 2 is a plot of the root mean square deviation (as a function of simulation time) of SH2 domain structures calculated during a computer simulation from the initial crystal structure.

FIGS. 3A-3C show an SH2 domain in complex with pTyr-Leu-Arg-Val-Ala, either by itself (FIG. 3A), superimposed with a representative structure from Cluster I of Example 1 (FIG. 3B), or superimposed with a representative structure from Cluster II of Example 1 (FIG. 3C).

FIG. 4 is diagram of a computer system useful for executing methods of the present invention.

### DETAILED DESCRIPTION

### I. Definitions

A “biopolymer” is any polymer that exists naturally in a living organism. Non-limiting examples of biopolymers include polypeptides, polynucleotides and polysaccharides.

A “body”, in the context of a component of a molecule, is defined as a unit of the molecule which is treated as a single mass or geometric structure for purposes of modeling the molecule. Accordingly, a body can be an individual atom of the molecule, a collection of atoms, or other abstract system of masses.

A “computationally-feasible order” refers to any order in which a particular sequence of tasks can be executed without altering the ultimate result. This concept is invoked because in some methods, the order of certain steps is not important so long as the steps are executed and the result is the same as if they were executed in the order originally presented.

The terms “conformation”, or “conformational variant”, when applied to a molecule or molecular structure, refer to a specific structure of the molecule that is one of several or many possible structures.

The term “dynamics”, when applied to molecules and molecular structures, refers to the relative motion of one part of the molecular structure with respect to another. Examples include, but are not limited to: vibrations, rotations, stretches, domain motions, hinge motions, sheer motions, torsion, and the like. Dynamics may also include motions such as translations, rotations, collisions with other molecules, and the like.

An “endogenous ligand” refers to any ligand that exists naturally in a living organism. Examples of endogenous ligands include growth factors, cytokines, neurotransmitters, calcium, vitamins, co-factors, small nucleic acids, peptides and the like.

A “flexible structure” is a structure of a molecule that has definite fixed coordinates for its constituent atoms or bodies, but that allows for a certain degree of internal motion about these coordinates, e.g., it may allows for bond stretching, rotation, etc. A dynamic pharmacophore is an exemplary flexible structure.

The term “in silico” refers to any method or process performed using a computer.

The term “in silico analysis” refers to any type of assay or analysis of molecular interactions performed on a computer. Non-limiting examples of in silico analysis include in silico screening, high-throughput in silico screening, in silico binding, in silico docking, in silico affinity determination, in silico molecular modeling, in silico annealing, in silico lead identification, in silico lead optimization, in silico ADMET, and the like. For example, running a set of docking simulation on a computer of various ligands binding to an active site of a protein constitutes an example of in silico screening.

Unless indicated otherwise, a “ligand” refers to any molecule known or suspected to bind another molecule. Its activity is detected and defined through its interaction with a target, such as a receptor, which specifically or non-specifically binds the ligand. Non-limiting examples of ligands include small molecules (e.g., organic and inorganic molecules), such as those conventionally used in small molecule therapeutics; other molecules such as endogenous ligands. Ligands may be classified in groups, such as agonists, antagonists, activators, inhibitors, catalysts and modulators of the activity of the target.

Unless indicated otherwise, a “molecule” is a representation of any microscopic structure formed of two or more atoms that are connected by chemical bonds. Non-limiting examples of molecules include representations of proteins (e.g., antibodies, receptors, etc), peptides, lipids, nucleic acids (e.g. natural or synthetic DNA, RNA, gDNA, cDNA, mRNA, tRNA, etc.), lectins, sugars (e.g. forming a lectin/sugar complex), glycoproteins, small molecules, organic compounds, monatomic or polyatomic structures such as salts, metals, etc.

A “polymer” is any organic molecule comprised of repeating subunits which are wholly or substantially similar in structure, or which have some molecular structure which is common to each sub-unit. Examples of polymers include biopolymers; polymer plastics (e.g., polyethylene, polypropylene, polystyrene, etc.); polymer fluids (e.g., polyethylene glycol, etc.), and the like.

The term “representation”, when applied to a molecule or molecular structure, refers to an abstract description of the molecule or molecular structure for use in a computer simulation. For example, one representation of a molecule is a set of coordinates which collectively defines the positions of atoms or bodies, or some abstract proxy thereof, constituting the molecule.

A “representative structure” is a molecular structure that is representative of a particular group or cluster of structures. A representative structure may be, for example, (i) a member of the group or cluster it represents that has a small (e.g., the smallest) deviation from the “average” of structures in that group or cluster; (ii) a hybrid of two or more structures from the group of cluster it represents, or (iii) a dynamics pharmacophore. A representative structure may be a rigid structure or a flexible structure.

A “rigid structure” is a static structure of a molecule that does not allow for internal bond rotations, stretching or other internal motions, e.g., a single abstract representation of a molecule which contains no dynamic information. A rigid structure may, of course, translate through space or a solvent as a rigid body.

A “small molecule” refers to any molecule that is not a polypeptide, polynucleotide or polysaccharide, and that has a molecular weight less than about 5 kDa. A small molecule is generally not a polymer, and can be a small organic molecule, or a representation of a lead compound, drug candidate, new chemical entity (NCE), metabolite, cytokine, a co-factor, e.g., a vitamin, etc. A small molecule can be derived from natural products or synthetic analogues developed and stored in combinatorial libraries.

A “target” refers to the molecule that is the primary subject of a method or simulation. For example, in an in silico screen of a set of ligands against a receptor, the receptor is the target.

### II. Introduction

A number of molecules, particularly biomolecules such as proteins, nucleic acids, lectins, sugars, etc., are dynamic entities undergoing a range of motions and conformational changes which are determined by innate properties of the molecules as well as various environmental factors, such as temperature, ionic strength, solvent properties, interactions with other molecules, and the like. Indeed, the structure or specific conformation of a protein is known to be linked its function. Many other molecules, particularly biomolecules, have similar relationships between structure and function. Thus, a complete understanding of molecular behavior, and a predictive capability based thereon, benefits from a simulation of as large a portion as possible of the full structural and dynamic space of a given molecule.

One way to describe and represent this molecular behavior is to use techniques and conventions of statistical mechanics (see, e.g., K. Huang, Statistical Mechanics, J. Wiley New York). For example, a set of canonical coordinates can be defined in a 6N-dimensional space via the following:

{q₁,q₂, . . . , q_{N};p₁,p₂, . . . , p_{N}}

where each qᵢ represent a three dimensional point in a generalized coordinate space (such as internal coordinates, e.g., torsion angle coordinates, or other convenient coordinates, such as Cartesian coordinates), and each pᵢ represent a three dimensional point in momentum space. 6N points are considered in the simulation—3N points for position and 3N points for momentum. If time is considered explicitly, then there is an additional point in the space, giving a (6N+1) dimensional representation space. One of skill in the art will appreciate that alternate representations may be chosen, such as generalized positions and velocities, energies, and the like.

Conventionally, this space is referred to as a representational space, and is often called a Γ-space. A single point in this space is referred to as a representational point. In the limit where N approaches infinity, a continuous density function may be used to represent the Γ-space, conventionally called the distribution function ρ(q, p, t), defined such that

ρ(q,p,t)d^{3N}pd^{3N}q

gives the number of representational points contained in the volume element d^{3N}pd^{3N}q at time t.

Thus, the coordinates, momentum, and the time dependence of both, are captured in the distribution function. Conventionally, the distribution function is normalized such that

$${\int }_{- ∞}^{+ ∞} {ⅆ}^{3 N} p {ⅆ}^{3 N} q ρ ( p , q , t ) = 1$$

For a given state {q,p}, the probability P of finding the system in an interval ε about that state is given by

$${\int }_{p}^{p + ϵ} {ⅆ}^{3 N} p {\int }_{q}^{q + ϵ} {ⅆ}^{3 N} q ρ ( p , q , t ) = P \{ q , p \}$$

Thus, the Γ-space can be sampled to find, for example, the most probable distributions of {q,p}, as indicated by the statistical weighting of the distribution function toward these configurations. One can also find structures that may not occur often, but nevertheless are quite important for activity or interaction. Examples of the latter include transition states—states which exist fleetingly between one stable conformational state and another and which may possess the appropriate structural and chemical properties to allow binding or other relevant interactions with another molecule.

### III. Discrete Points in the Representational Space—Defining Conformational Variants and Representative Structures of a Molecule

In many cases, the continuous distribution described above may be approximated by a discrete set of points, which optionally allows, for example, a density of states sufficiently high to use for various applications detailed herein and/or to make interpolation between points possible. In such a computationally-derived distribution function, a series of structural conformational variants (which may include dynamic information) are generated for a given molecule. These structural variants may subsequently be utilized to characterize interactions with exogenous molecules, e.g., ligands. The distribution function may also be calculated to include the effects of a given exogenous molecular structure on a target molecule. In either case, the distribution function is generated computationally by keeping track of the positions and momenta (or velocities) of the 6N canonical coordinates, and developing an archive of these values.

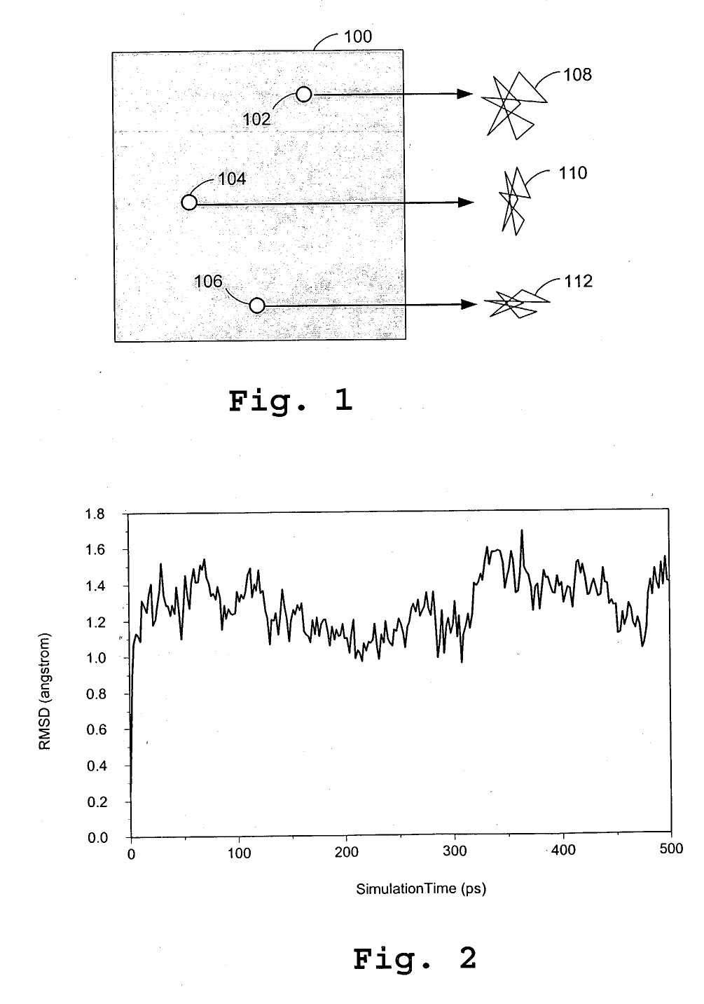

FIG. 1 shows a 2-dimensional graphical abstraction 100 of such a computationally-derived distribution function for a representational space of a selected protein molecule. Each point in the Three “points” 102, 104 and 106 in the representational space, corresponding to three conformational variants (108, 110 and 112, respectively) of the molecule are identified. Using methods detailed herein, the practitioner can generate a set of such structures and conformations of an isolated molecule to produce a tangible & useful approximation of the complete representational space. This set of structures or conformational variants, together with associated dynamics properties if so desired, may then be “cataloged” for future use in the study of the molecule.

One method of generating a discrete set of structures which constitute a representational space is based on a consideration of the statistical properties of the system. These statistical properties are, in turn, based upon more fundamental properties, which can be described by the equations of motion for the system. The equations of motion are a set of differential equations which constrain the system to obey certain physical laws, as well as describing the fundamental forces acting on the system. A set of such equations, commonly used in the art, is as follows:

$$\frac{{ⅆ}^{2} q}{ⅆ {t}^{2}} - F ( q ) = 0$$

In this equation, F is the force acting on the system at the coordinate q. For a system with N coordinates, the following is a generalized form of this equation:

$${\sum }_{i = 1}^{N} \frac{{ⅆ}^{2} {q}_{i}}{ⅆ {t}^{2}} - F ( {q}_{i} ) = 0$$

Newtonian mechanics can be applied to such a system with pre-defined coordinates and initial conditions, and solved for each of the coordinates q to yield the following relationships:

$${q}_{i} ( t ) = position$$

$$m {\dot{q}}_{i} ( t ) = m \frac{ⅆ {q}_{i}}{ⅆ t} = {p}_{i} = momentum$$

$${\ddot{q}}_{i} ( t ) = \frac{{ⅆ}^{2} {q}_{i}}{ⅆ {t}^{2}} = acceleration$$

Thus, by using realistic values (or good approximations thereof) for the forces acting on a system, and solving the above differential equations, it is possible to generate a representational space. This space is comprised of the values for each of the coordinates above, and is given by:

{qᵢ(t),pᵢ(t)}=representational space

Another common method of generating the representational space is to use the Euler-LaGrange equations, which are given as follows:

$$L = T - V$$

$${\sum }_{i} [ \frac{ⅆ}{ⅆ t} ( \frac{\partial L}{\partial {\dot{q}}_{i}} ) - \frac{\partial L}{\partial t} ] = 0$$

Here, the Lagrangian L is defined to be the difference of the kinetic energy T and the potential energy V acting on the system; and the second equation is a result of the requirement that the Lagrangian must be an extremum of the system.

A salient point in both of these cases is that a representational space is generated for a finite set of discrete structures using fundamental physical principles and forces acting on the system. The above equations are solved by using an integration procedure which is appropriate to the solution desired. A computational approach is typically used to integrate the equations of motion, wherein the integrals are solved numerically, subject to the appropriate boundary and initial conditions, to yield numerical solutions.

A. Generating a Representational Space and Assessing Interactions

The approaches described above can be implemented by generating a representational space for the molecule, e.g., protein of interest. The initial specification of the protein may take any form suitable and amenable to the computational methods described herein, examples of which include, but are not limited to: putative or known primary structure (the sequence of amino acids in the protein); putative or known secondary structure (the local structures of the protein, such as helices and hairpins); one or more representations of the tertiary structure (the three dimensional shape of the protein), such as one or more conformations, either known (for example, as obtained from NMR or X-ray crystallography) or predicted; one or more representations of the quaternary structure (the three dimensional shape of two or more proteins in a complex, wherein each protein is associated with the complex in such a way that the complex as a whole has a characteristic shape and function), such as one or more conformations of the quaternary structure, either known or predicted. The result of this specification may be comprised of a set of coordinates q and optionally velocities u designed to represent the protein at some initial simulation time.

The solvent conditions for the molecule of interest are also typically specified. They may take the form of an implicit solvent model, an explicit solvent model, some combination of the two, or any other method which will render a suitable environment in which to model the protein. The choice of solvent is made on the basis of computational tractability, physiologic relevance, and the goal of the simulation. The solvent model may optionally include necessary properties for protein functionality, such as co-factors, ligands, counter-ions, pH properties, other proteins and peptides, nucleic acids, lipids, saccharides, and the like.

After the protein and its environment have been specified, the methods described previously are used to generate a time-dependent set of coordinates for the protein. For each given point in time of the simulation, there is a corresponding set of coordinates

Γᵢ={q(tᵢ),u(tᵢ)}

The set of all coordinates taken at one given time comprise one representation of the protein. The set of all coordinates, taken over all time, comprise the full representation of the protein

Γ={Γᵢ }

In this particular embodiment, each molecular structure being simulated will have its own distinct representation. For example, if one desires to simulate the interaction between two proteins, then each protein will have a distinct representation Γ. Also in this particular embodiment, the contents of each representational space are distinct and independent of the contents in the representational spaces of other molecular structures in the simulation. In other word, this embodiment does not consider the effect of one molecular structure on a second molecular structure.

Once generated, the representational space may then optionally be used to create a distribution function, as described previously, wherein the statistical likelihood of the existence of one or more representative structures or conformations may be derived. The distribution function, or proxy thereof, may then be used to determine which structures and conformations are accessible to the protein, and thus which structures and conformations may be involved in an interaction.

Upon specifying a set of structural representations of the protein, representations of one or more ligands are selected for study vis-a-vis their interactive properties with the protein. One of skill in the art may base the choice of representation and ligand on a number of different criteria, including but not limited to: known or suspected binding properties of the ligand to the protein; known or suspected physiologic or pharmacologic properties of one or more ligands; known or suspected structure-activity relationship(s) of one or more ligands; class properties of one or more ligands, including but not limited to, chemical scaffolds known to, or suspected of, participating in desired pharmacologic effects, and chemical motifs which are known to, or suspected of, participating in desired pharmacologic effects; and the like.

After an initial set of structural representations of the protein and the ligand(s), and the evaluation criteria for the interaction(s) of the protein with each of the ligands, are selected, the practitioner next chooses a method for studying the interactions. In one embodiment of the present invention, the interaction of a specific ligand with each of the representations of the protein is studied using one or more of the existing molecular docking programs. Examples of such programs include, but are not limited to: AMBER (http://www.amber.ucsf.edu/amber/amber.html), AMMP (http://www.cs.gsu.edu/˜-cscrwh/ammp/ammp.html), CHARMM (http://yuri.harvard.edu/), Dalton Quantum Chemistry Program (http://www.kjemi.uio.no/software/dalton/dalton.html), Deep Viewer (http://expasy.cbr.nrc.ca/spdbv/), FTDock (http://www.bmm.icnet.uk/docking/), TINKER (http://dasher.wustl.edu/tinker/), and the like. In this embodiment, each representation of the protein is treated as a fixed structure, against which the commercially available docking program performs its functions directed at determining the nature of an interaction between a given ligand and a given protein. The endpoint of such a study is based on the goals of the study. For example, the goal may be to determine which ligands are likely to bind a given protein, and/or to determine what the affinity is between a given ligand and a given structure of a protein. In addition, other goals consistent with determining which ligands hold potential therapeutic value may optionally be sought. Examples of the latter include: finding particular conformational variants which are known or suspected to play a role in the physiologic functioning of the target, finding ligands which bind specific transition states, which may exist for very short times, but nonetheless be important in the formation of physiologically active or inactive states.

B. Molecular Dynamics

An exemplary method for generating a discrete set of structures which collectively define the representational space of a selected molecule is through the use of a molecular dynamics simulation. Such simulations are well known in the art. See, e.g., T. Schlick, Molecular Modeling and Simulation—an Interdisciplinary Guide, New York, 2002, Springer Verlag; A. Leach, Molecular Modeling—Principles and Applications (2^{nd} ed.), Dorchester, England, 2001, Prentice Hall/Dorset Press; and Rapaport, D.C., The Art of Molecular Dynamics Simulation, Cambridge, England, 1995, Cambridge University Press. In essence, a molecular dynamics model simulates the movement of real molecules by repeatedly moving the atoms of the system under the influence of forces. After each movement of the modeled molecule(s) to a new conformation, the forces are redetermined and the bodies or atoms are again moved, but this time under the influence of the new forces. These methods are typically implemented via a computer, such that the relevant algorithms are coded in one or more computer software programs and executed on a computer or a group of computers.

In methods of the invention utilizing molecular dynamics simulations, a representation of a molecular system is initially prepared, typically in a computer usable form. The molecules which form the molecular system being modeled are typically partitioned into a set or sets of bodies, which may be either individual atoms, or collections of atoms connected to one another, or more abstract representations such as mass and/or geometry-carrying units.

The bodies are defined with respect to a selected set of generalized coordinates which collectively express the locations and orientations of these bodies, and provide a reference frame for calculating kinetic properties, such as momentum and/or velocity and/or acceleration. The coordinates may be any convenient but mathematically complete coordinate system. Examples include Cartesian coordinates (i.e. the orthogonal x, y, z locations and velocities of the bodies in space), or the coordinates may be internal coordinates of the system, e.g., torsion angle, coordinates, where the relative displacement between the bodies (but not their absolute position) is tracked, along with the (absolute) x,y;z spatial location of one of the bodies, designated the base body. Other representations are also possible, e.g., the Cartesian rigid body approach described in Turner, et al., U.S. Pat. No. 5,424,963.

The bodies are typically held in relationship to one another by covalent bonds, which constrain the conformation space that can be adopted by the molecule, but also allow for certain movements and rotations of the bodies relative to one another. The covalent bonds, as well as non-covalent interactions (including hydrogen bonds, van der Waals interactions and electrostatic interactions) may be expressed directly, such as specifying the electrostatic fields of molecular or atomic charges; or as potential energy surfaces or volumes, (such as the Lennard-Jones potential), the gradients of which result in forces on the individual bodies making up the molecule being modeled. These forces make up the “intra-solute” forces at work “within” a solute molecule, and the inter-solute forces between solute molecules. In addition to the above-described intra- and inter-solute forces, the direct effects of implicit solvent on the solute may be expressed as impulse functions, forces, or as energy functions which result in forces.

The forces are used in differential equations which describe the kinetics of the molecule or system being modeled. For multibody systems, these equations are generally solved using numerical integration methods (see, e.g., PCT publication numbers WO02/39087 (Sherman & Rosenthal; Method for Large Timesteps in Molecular Modeling); WO02/36744 (Rosenthal; Method for Residual Form in Molecular Modeling); and WO02/061662 (Rosenthal; Method for Analytical Jacobian Computation in Molecular Modeling)). Any of a number of known integrators may be used for numerical integration—see, for example, Hairer, et al., Solving Ordinary Differential Equations I, 2^{nd} ed, Springer Verlag, Heidelberg, (1993); and Hairer and Wanner, Solving Ordinary Differential Equations II, Springer Verlag, Heidelberg (1991).

C. Generating a Set of Conformational Variants

According to one aspect of the invention, a target molecule is selected and used as a basis for the generation of a set of conformational variants, i.e., a set of structures or conformations which the target can adopt. In one embodiment, the target is a biomolecule, e.g., a protein, such as a receptor or other target for potential therapeutic intervention. In a preferred embodiment, the target molecule is a protein.

Exemplary proteins to which methods of the invention may be applied include, without limitation, all varieties and types of receptors; viral, prokaryotic and eukaryotic, proteins; integral membrane proteins, such as the photosynthetic reaction center, bacteriorhodopsin, G-protein-coupled receptors, nicotinic acetylcholine receptor, proton and ion pumps, ion channels, and the like; fibrous and structural proteins, such as actin, myosin, dystrophin, troponins, F- and G-actin, dynein, microtubules, ankyrin, vilin, collagen, fibronectin, laminin, vitronectin, fibrinogen, fibrin, titin, glycoproteins, keratin, and the like; enzymes, such as transferases, hydrolases, lyases, isomerases, dehydrogenases, and the like; nucleic acid-related proteins, such as polymerases, nucleases, ligases, gyrases, topo-isomerases, DNA-binding proteins, transcription factors, zinc-fingers, repressors, histones, steroid receptors and the like; response elements, such as kinases, phosphatases, G-proteins, calmodulin, lipases, adaptors ( SH2, SH3, PH domains), and the like; redox and electron transport proteins, such as dismutases, cytochromes, thioredoxin, ferredoxin, and the like; hydrolases, such as DNases, RNases, lipases, glycosidases (e.g., lysozyme), and proteinases (e.g., viral proteases, such as HIV protease, serine proteases, aspartic proteases, cysteine proteases, and zinc metallo-proteases); binding proteins, such as albumin, retinol-binding protein, ferritin, hormones (e.g., insulin), cytokines, growth factors immunoglobulins, lectins, and the like; periplasmic proteins, such as arabinose-binding protein, sulphate-binding protein, phosphate-binding protein; various types of foldases and chaperone proteins, and others. In embodiments where the molecule is a peptide or protein, the peptide or protein may comprise, for example, between about 5 and 10 amino acid (“aa”) residues; between about 10 and 25 aa residues; between about 25 and 50 aa residues; between about 50 and 100 aa residues; between about 100 and 250 residues; between about 250 and 500 residues; between about 500 and 1000 residues, and over about 1,000 residues. Alternatively, the peptide or protein may comprise at least about 10 aa residues; at least about 25 aa residues; at least about 50 aa residues; at least about 100 aa residues; at least about 150 aa residues; at least about 300 aa residues, at least about 500 aa residues, or at least about 1000 aa residues.

Starting points for generating conformational variants of the invention may be, e.g., crystal structures, for example, obtained as “pdb” files from the Protein Data Bank, which contains over 20,000 structures (see http://www.rcsb.org/pdb/index.html; H. M. Berman, et al., “The Protein Data Bank” Nucleic Acids Research 28:235-242 (2000)); structures may also be obtained from NMR studies, from predicted structures, or other sources as described above.

In a preferred embodiment, the molecule has a binding site for a ligand. Suitable ligands include, but are not limited to, antigens, nucleic acids (e.g. natural or synthetic DNA, RNA, gDNA, cDNA, mRNA, tRNA, etc.), lectins, sugars, oligosaccharides, growth factors, cytokines, small molecules such as drug candidates (from, for example, a random peptide library, a natural products library, a legacy library, a combinatorial library, an oligosaccharide library or a phage display library), metabolites, drugs of abuse and their metabolic by-products, enzyme substrates, enzyme inhibitors, enzyme co-factors such as vitamins, lipids, steroids, metals, oxygen and other gases found in physiologic fluids, cells, cellular constituents, cell membranes and associated structures, cell adhesion molecules, natural products found in plant and animal sources, other partially or completely synthetic products, and the like.

The set of conformational variants may be generated using structures, or running simulations, which include a ligand bound at the ligand binding site. The presence of a ligand in such co-structures enables more accurate modeling in situations where “induced fit” plays an important role, i.e., in cases where the binding of the ligand causes a conformational change in the structure of the target molecule. In these cases, structural and dynamic properties which are unique to the ligand-target co-structure may be noted and archived for later use. Particular properties of ligand binding may optionally include: novel or unique structures which alter steric effects during binding; inducement or repression of structures known to, or suspected of, cause biologic or physiologic effects; alterations in any part of the target which my enhance or inhibit secondary binding, e.g. to co-activators, co-repressors, or multimeric complex formation, which is known to, or suspected of, playing a role in physiology. In performing subsequent screens against conformational variants generated with a ligand in place, the ligand that was present in order to generate the set of conformational variants is “removed” from the structure, and new putative ligands, small molecules or other compounds are screened in silico for binding or docking in the pocket created by the removed ligand. In addition, subsequent screens may select for one or more structures or representations which may be physiologically or pharmacologically relevant, based on one or more criteria, as exemplified by the previous examples.

Any of a number of approaches may be used to generate a set of conformational variants according to the present invention. In one embodiment, the set is assembled from crystal structure, nuclear magnetic resonance (NMR), or other empirical data. For example, NMR is capable of generating data which can be used to determine the structure of a variety of molecules. Often, data are available on a number of different conformational variants of the same molecule. In such cases, a set of conformational variants of a particular molecule may be assembled by collecting together structures provided by NMR data on that molecule. This set can them be subjected to additional steps according to methods of the invention as specified herein. Alternatively, one or more X-ray crystal structures may be available for a particular molecule. These structures may also be assembled as described above to form a set of conformational variants for that particular molecule, and used in methods of the invention as detailed herein.

In another embodiment, a representational space is formed using a molecular modeling method as described above, to generate the set of conformational variants. For example, a molecular dynamics simulation may be run for a selected period of time to generate a set of “snapshots” of structures during the simulation. These snapshots may then be pulled together into a set of conformational variants for use in subsequent steps of methods of the invention. Such a molecular dynamics simulation may be run for any suitable length of time, e.g., for a few pico-seconds to tens or hundreds of picoseconds, or longer, for durations in the range of nanoseconds, e.g., 1 ns, 10 ns, 100 ns, or into microseconds or longer. One of skill in the art can determine a suitable duration for the run based on factors such as the amount of computational resources available, the amount of time that can be allotted per run, the complexity of the protein and/or the representational space it defines, and the like. Alternatively, the simulation may be run until a predetermined number of distinct structures or conformational variants are generated, until a defined portion of the representational space available for the protein has been explored, or based on other considerations readily apparent to one of skill in the art.

An energy minimization approach, such as Monte Carlo modeling, may also be used to generate the set of conformational variants. In this case, a number of factors may be weighed in deciding how long to run the minimization. For example, the minimization may be run for a pre-determined period of time, until a certain minimum energy level is reached, until a predetermined number of structures having an energy level below a certain threshold are achieved, and the like.

As mentioned above, in any molecular modeling-based approach for generating structures, a set of conformational variants may be generated by simply storing “snapshots” of the evolving or changing structure at points in time during the run. For instance, in Example 1, a simulation was run for 500 ps, and structures were saved every 2 ps, generating a set of 250 conformational variants. The number of structures in a set of conformational variants is determined by the practitioner based on computational efficiency, the dynamic properties of the target molecule (e.g., does the molecule adopt a wide range of structures during the simulation—suggesting a need for a larger number of structures in the set—or only a limited number—suggesting that fewer structures may be sufficient. In general, the number of structures will range from a few to tens, hundreds, thousands or more. In one embodiment, the set contains between about 10 and about 100 structures. In another embodiment, the set contains between about 100 and about 1000 structures; in yet another embodiment, it contains between about 1000 and 10,000 structures. Note that conformational variants generated in this manner are not necessarily unique, in that one or more variants in the set may have the same or essentially the same structure. However, this does not affect the usefulness of the method, since the same or highly similar variants will simply be sorted into the same cluster in a subsequent step of the method.

D. Forming Clusters of Conformational Variants

After generating a set of conformational variants of a selected molecule, the variants are clustered using any of a variety of suitable clustering or grouping algorithms. Clustering algorithms are known in the art and are typically based on a measure of proximity or distance between data points (see, e.g., Jain and Dubes (Practice Hall, Anglewood Cliffs, N.J., 1988)). Clustering algorithms may be applied to the entire molecule, or to one or more portions of the molecule. For instance, the clustering algorithm may be applied only to amino acid residues in the vicinity of a ligand binding site, as is illustrated in Example 1, below.

A cluster analysis typically employs a measure of the similarity (or dissimilarity) between pairs of objects. When comparing molecular conformations, the root mean square difference (RMSD) of the Cartesian coordinates of the molecular system can be used to measure the distance between two conformations. Alternatively, internal coordinates (torsion angles) may be also used. In this case one can either calculate the Euclidean distance dᵢⱼ between conformations:

$${d}_{ij} = \sqrt{{\sum }_{m = 1}^{{N}_{tor}} ( {ω}_{m , i} - {ω}_{m . j} )}$$

or the Manhattan distance:

$${d}_{ij} = {\sum }_{m = 1}^{{N}_{tor}} \vert {ω}_{m , i} - {ω}_{m . j} \vert$$

(Leach A. R. Addison Wesley Longman Limited, England, 1996). In each case, ω_{m,i} is the value of torsion angle m in conformation i and ω_{m,j} is the value of torsion angle m in conformation j. Nₜₒᵣ is the total number of torsion angles.

Any of a number of known clustering techniques, methods, approaches or algorithms may be used in connection with the present invention. Exemplary clustering techniques include (i) partitioning around medoids, and (ii) hierarchical algorithms. Many such clustering algorithms refer to “objects” which are clustered. In the context of the present invention, such objects are typically structures or conformational variants, which are submitted to the clustering algorithms for clustering.

A widely used set of approaches from the hierarchical clustering methods is collectively termed linkage clustering. Linkage or pairwise methods are particularly suitable for the clustering of molecular structures. To use this method, one typically first determines the distance between each pair of conformations. At the start of the clustering, each data set contains as many clusters as there are conformational variants, with each cluster containing a single conformational variant. At each step of the clustering, the total number of clusters is reduced by merging the most similar pair of clusters into a single cluster. Clustering continues until the number of clusters falls below a specified maximum number, or until all the conformations have been merged into a single cluster. Such a way of clustering is referred to as an agglomerative method. A non-limiting example of an agglomerative method is agglomerative nesting (AGNES; Kaufman and Rousseeuw, 1990, chapter 5). These algorithms are simple to program and produce a clustering that is independent of the order in which the objects are stored. (Leach A. R. Addison Wesley Longman Limited, England, 1996). Conversely, divisive clustering methods start out with a single cluster containing all of the data, which is than portioned into clusters. An example of divisive method is divisive analysis (DIANA; Kaufman and Rousseeuw, 1990, chapter 6).

Another clustering algorithm is based on partitioning around medoids (PAM) (Kaufman and Rousseeuw, 1990, p. 164). Here, one obtains k clusters and the method selects k objects, referred to as representative objects, in the data set. The corresponding clusters are then found by assigning each remaining object to the nearest representative object. Not every selection of k representative objects gives rise to a good clustering. Representative objects are preferably chosen so that they are centrally located in the clusters they define. More specifically, the average dissimilarity of the representative object to all the other objects of the same cluster is minimized. Thus, an optimal representative object is called the medoid of its cluster. PAM sometimes produces hard clustering, because it makes a clear-cut decision for each data point. Fuzzy clustering, on the other hand, allows for a certain amount of ambiguity in the data, which is appealing because it allows a description of some of the uncertainties that accompany “real” data sets (Kaufman and Rousseeuw, 1990, p. 164. An example of fuzzy clustering is the FANNY algorithm ((Kaufman and Rousseeuw, 1990, p. 164). Although fuzzy clustering yields much more detailed information on the structure of the data than hard clustering, the amount of output generated using the fuzzy clustering approach grows rapidly with the number of objects and number of clusters, and may render the method impractical for use with large data sets. However, this latter limitation can be mitigated through the use of discrete-to-continuous transformations, e.g. linear or non-linear interpolation or extrapolation of discrete sets into continuous sets.

A number of other clustering methods may be employed, including, e.g., central clustering (which implicates K-means and its generalizations) and mixture modeling for density estimation (which implicates Bayesian & minimum description length). Other clustering methods are described, e.g., in Jain and Dubes (Practice Hall, Anglewood Cliffs, N.J., 1988), by Sokol (Sokol R. R., J. Van Ryzin, Ed., Academic Press New York, 1977, p. 1); Zupan (Zupan J., John Wiley & Sons, New York, 1989); Agrafiotis, et al., U.S. Pat. No. 6,453,246, “System, method, and computer program product for representing proximity data in a multi-dimensional space”; L. Hunter (1999) “Statistical clustering approaches and their use for classifying molecular structures” at http://www.science.gmu.edu/˜-lhunter/talks/ericel/; and “An experimental Comparison of Several Clustering and Initialization Methods”, at ftp://ftp.research.microsoft.com/pub/tr/tr-98-06.ps.

The clustering of an exemplary set of conformational variants is described with respect to Example 1, below. A Fuzzy Analysis clustering algorithm was applied to conformational variants of the complex between the phosphotyrosine recognition domain of SH2 of v-src and tyrosine-phosphorylated peptides. The data suggest that three positively charged residues, Arg155, Arg175 and Lys203, interact with the phosphotyrosine moiety through salt bridges and hydrogen bonding and aromatic-cation interactions (Waksman et. al. (1992) Nature 258:646-653). Furthermore, the data suggest that Ser177 and Thr179 form hydrogen bonds with phosphate oxygens. Based on these results, one may conclude that these interactions synergistically are responsible for the binding of the phosphotyrosine moiety.

E. Selecting Representative Structures from the Clusters

Once a suitable clustering algorithm has been applied to the structures or set of conformational variants, and a suitable group of clusters has been identified, the practitioner identifies a representative structure from the cluster which can be used in any subsequent steps, such as in silico analysis, e.g., screening, of putative ligands against the target molecule. Any of a number of known approaches may be used to select a representative structure. In one embodiment, the structure representing a particular cluster is one of the structures contained in that cluster. Typically, the representative structure is one that has a small (e.g., smallest) deviation from the “average” structure which defines that particular cluster, and is thus “representative” of the cluster. In another embodiment, the representative structure from a particular cluster is a hybrid structure representing two or more structures contained in that cluster.

Alternatively, the representative structure may be a dynamic pharmacophore model developed using conformational variants within the cluster. The development and application of dynamic pharmacophore models is known in the art (see, e.g., Carlson, et al., (2000) J. Med. Chem. 43:2100-2114). In brief, a dynamic pharmacophore model is a representation of all or most structures in a particular cluster. It accounts for the inherent flexibility of portions of the portions of the molecule that are being modeled (e.g., the active site or ligand binding site), by identifying residues at that site whose positions are conserved among members of that cluster. Residues that vary in position among the members of a particular cluster are treated as “flexible”, and thus do not constrain ligand fitting to the same extent as the “conserved” residues in the dynamic pharmacophore model. A dynamic pharmacophore model is thus an exemplary flexible representative structure.

### IV. Using Representative Structures for in Silico Analysis

The present invention finds application, inter alia, to a broad host of in silico analysis methods, including in silico screening (screening putative ligands and studying potential drug candidate compounds, such as lead compounds, new chemical entities (NCEs), and the like, for purposes of optimizing therapeutic efficacy), high-throughput in silico screening, in silico binding, in silico docking, in silico affinity determination, in silico molecular modeling, in silico annealing, in silico lead identification, in silico lead optimization, in silico ADMET, and the like. In particular, the present invention may be used in studies addressing the selectivity or specificity of ligand binding/activation/inactivation of related receptors, e.g., structurally-related receptors from the same molecular family that have different functions (e.g., different physiological functions).

Representative structures may be used, for example, as the target structures in virtual ligand screening (VLS) of small molecules docking into target active sites. A number of such in silico analysis or virtual screening methods are known in the art. For example, a number of commercial docking software programs are available for performing docking and other types of simulated binding of ligands to target structures. Examples of such programs include, but are not limited to: AMBER (http://www.amber.ucsf.edu/amber/amber.html), AMMP (http://www.cs.gsu.edu/˜-cscrwh/ammp/ammp.html), CHARMM (http://yuri.harvard.edu/), Dalton Quantun Chemistry Program (http://www.kjemi.uio.no/software/dalton/dalton.html), Deep Viewer (http://expasy.cbr.nrc.ca/spdbv/), FTDock (http://www.bmm.icnet.uk/docking/), TINKER (http://dasher.wustl.edu/tinker/), DOCK (http://www.cmpharm.ucsf.edu/kuntz/dockinfo.html), GOLD (http://www.ccdc.cam.ac.uk/prods/gold/), FlexX (http://cartan.gmd.de/flexx/), AutoDOCK (http://www.scripps.edu/pub/olson-web/doc/autodock/), MCDOCK (Ming Liu & Shaomeng Wang, “A Monte Carlo simulation approach to the molecular docking problem”, Journal of Computer-Aided Molecular Design, 13:435-451, 1999.), ProDock (J.-Y. Trosset and H. A. Scheraga. “PRODOCK: Software Package for Protein Modeling and Docking” J. Comp. Chem., 20(4):412-427, 1999).

In addition, in silico lead optimization techniques may be employed concurrently with the methods described herein. Many of these techniques are well-known to those skilled in the at of medicinal chemistry, and include: additions to the ligand scaffold or groups to add more bonding potential to the ligand-target complex, additions and/or deletions to the ligand to favorably alter steric effects during the binding process, additions and/or deletions to the ligand to favorably alter the co-structure of the ligand-target complex, and the like.

Methods of the invention provide a number of advantages over currently used in silico analysis methods. For example, in contrast to using a single crystal structure for in silico analysis, one can test against a group of representational variants of a particular target molecule that collectively represent the a full or partial set of conformations that can be adopted by that target. In silico docking, binding or screening to such a collection of representational variants is therefore much more likely to faithfully simulate real physiologic conditions and processes than approaches which do in silico docking, binding or screening against only a single or a small group of X-ray crystal or NMR structures. Further, because the representative structures generated using methods of the invention are only a small fraction of the total number of structures generated by a typical molecular dynamics simulation, in silico analysis can be performed much more efficiently on a larger number of compounds than would be possible if every structure generated by the molecular dynamics simulation were used. And lastly, it is self-evident that methods of the invention are much more efficient at high-throughput in silico screening than running a separate molecular dynamics trajectory with every ligand for which in silico data are desired.

Methods of the invention are also more accurate that doing in silico screening on a subset of structures generated by random or regular sampling from a molecular dynamics simulation. For example, although one could generate a subset of structures for in silico analysis by selecting, e.g., every 50^{th} structure generated in a molecular dynamics simulation, such an approach could (i) easily miss transient, but structurally distinct, conformations that may be important to ligand binding or other interactions in vivo, and (ii) waste resources by repeatedly screening against essentially the same structure, representing a state in which the target resides for a large fraction of the simulation. In contrast, methods of the present invention are designed to generate a collection of unique (relative to one another) structures that together represent all, a substantial portion of, or a selection of the most physiologically relevant, distinct conformational states adopted by the molecule, thus providing a higher-quality data set for in silico analysis and in silico lead optimization.

### V. Computer System

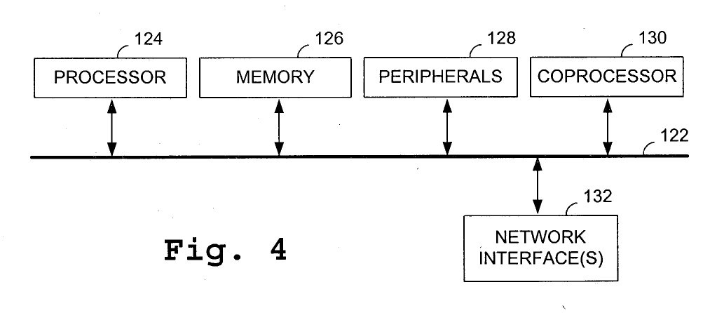

To carry out the calculations described above, a computer system may be used with at least one processor and associated memory subsystem for holding the computer code to instruct the processor to perform the operations described above. FIG. 4 illustrates the basic architecture of such a computer system having a processor 124, a memory subsystem 126, peripherals 128 such as input/output devices (keyboard, mouse, display, etc.), perhaps a co-processor 130 to aid in the computations, and network interface devices 132, all interconnected by a bus 122. The memory subsystem optimally includes, in increasing order of access latency, cache memory, main memory and permanent storage memory, such as hard disk drives. Given the amount of intensity of computation, it should be understood that the computer system could include multiple processors with multiple associated memory subsystems to perform the computations in parallel; or, rather than having the various computer elements connected by a bus in conventional computer architecture as illustrated by FIG. 4, the computer system might formed by multiple processors and multiple memory subsystems interconnected by a network.

The following example illustrates but in no way is intended to limit the present invention. cl EXAMPLE 1

### Cluster Analysts of the Phosphotyrosine Binding Pocket of SH2-VRC

Generating a Set of Conformational Variants

Langevin dynamics simulation was generated using the Imagiro 1.0 program on the apo SH2 domain of v-src oncogene product (Waksman et. al. (1992) Nature 258:646-653) (pdb access code: 1spr) at 300 K. Forces on the atoms were calculated on the basis of the AMBER force field (Cornell et. al. (1995) J. Am. Chem. Soc., 117:5179-5197; Kollman et al. (1997) in “Computer Simulation of Biomolecular Systems”, W. F. van Gunsteren, et al, eds., Vol. 3, p83-96). The GB/SA continuum model (Still et. al. (1990) J. Am. Chem. Soc. 112:6127-6129) was used to model the aqueous environment. No cutoffs were used for non bonded interactions. All protein atoms were propagated according to the Langevin equation using Runge-Kutta-Merson 5 integrator with 0.01% accuracy. The initial structure was subjected to a 100 step of Quasi-Newton minimization to relieve energetically unfavorable contacts in the protein. Next, a Langevin dynamics simulation was performed for 500 ps at 300 K. γ was set to 91 ps⁻¹ to mimic the viscosity of water. Structures were saved every 2 ps, generating a set of 250 conformational variants.

FIG. 2 shows the root mean square deviation (RMSD) of the calculated structures from the initial crystal structure (the heavy backbone atoms of the SH2 domain) as a function of time. Note that equilibrium is reached after about 25 ps of simulation.

Forming Clusters of Conformational Variants

The set of 250 conformational variants was clustered using a fuzzy analysis algorithm (a generalization of the partitioning method of Kaufman and Rousseeuw, (1990) “Finding Groups in Data: An Introduction to Cluster Analysis” Wiley-Interscience, New York (Series in Applied Probability and Statistics), 342 pages). The fuzzy analysis clustering method was used to find related groups of conformations on the basis of their pairwise dissimilarities. Dissimilarities between conformations were defined by calculating the root-mean-square (rms) Cartesian deviations of the selected atoms of superimposed structures in the trajectory and the crystal structure according to the following formula

$$\sqrt{\frac{1}{n} {\sum }_{i = 1}^{n} {\vert {x}_{i} - {Tx}_{i}^{′} \vert}^{2}}$$

where n is the number of atoms, xᵢ is the Cartesian coordinate of the ith atom, and T is the transformation matrix that best superimposes the two structures (Troyer and Cohen (1995) Proteins: Structure, Function and Genetics 23:97-110). Dissimilarities were calculated using the C(ζ) atom in Arg155 and Arg175, the O(γ) atom in Ser177 and Thr179, and the N(ζ) in Lys203. The dissimilarity matrix was constructed using a Perl script. The dissimilarity matrix was used as input file for the Fuzzy Analysis clustering program FANNY (Kaufman and Rousseeuw, 1990, Op.Cit.). A minimum of two and a maximum of ten clusters were selected to be generated in the FANNY program.

The number of clusters into which the conformation can be divided was selected on the bases of the silhouette width function, sᵢ. The value of the silhouette width of an individual conformation can be between −1 and 1. It is defined by the average dissimilarity, aᵢ, between conformation i and all other conformations within cluster A and the average dissimilarity, bᵢ, between conformation i and all other conformations within the neighboring cluster B. Therefore,

sᵢ=1−aᵢ/bᵢ if aᵢ<bᵢ

sᵢ=0 if aᵢ=bᵢ

sᵢ=bᵢ/aᵢ−1 if aᵢ>bᵢ

In the present Example, the optimal clustering of the system was selected on the basis of the highest average silhouette with of all clusters (Kaufman and Rousseeuw, 1990, Op.Cit.; Watts et. al., (2001) J. Mol. Struct. 535:171-182).

The number of clusters representing the optimal clustering of the system is based on the highest average silhouette width of all clusters. The highest average silhouette width of all clusters is defined as the silhouette coefficient of the system. Silhouette width values between 0.71 and 1.00 represent a strong cluster, 0.51 and 0.70 a reasonable cluster, and 0.26 and 0.50 a weak cluster that could be artificial and less than 0.26 no cluster.

A clustering analysis was performed on the side chains of Arg155, Arg175, Ser177, Thr179 and Lys203 to find the main conformational families of the binding pocket of the apo-SH2 domain of v-src, yielding two separate clusters. The average silhouette with of the entire data set was 0.43. Cluster I had 234 members and a silhouette width of 0.42, while Cluster II had 16 members and a silhouette width of 0.51.

Selecting Representative Structures

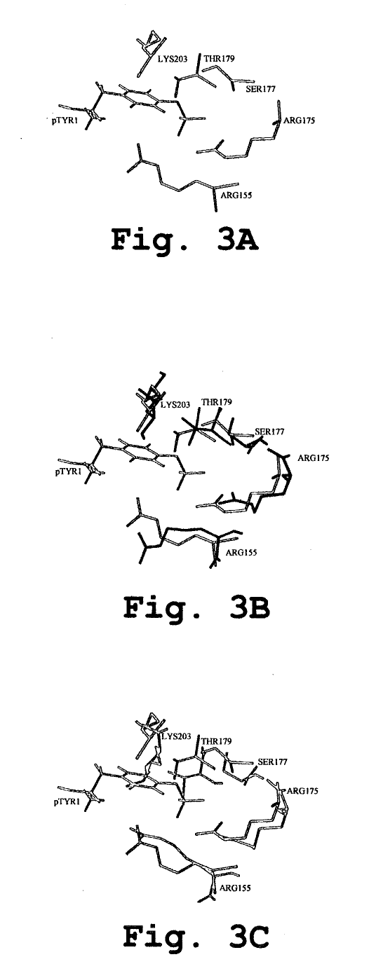

Representative structures from Clusters I & II (those structures with the highest silhouette coefficient from each cluster) are shown in FIGS. 3A-3C superimposed on the SH2 domain crystal structure complexed with pTyr-Leu-Arg-Val-Ala. Only selected residues in the SH2 binding pocket and the pTyr are shown. FIG. 3A shows the crystal structure of the complex (Waksman et. al. (1992) Nature 258:646-653, pdb access code: 1shb, SH2 domain in red and pTyr in cyan); FIG. 3B shows the crystal structure of the complex and Cluster I (Cluster I in blue); FIG. 3C shows the crystal structure of the complex and Cluster II (Cluster II in green). As can be appreciated from the data, although structures in Cluster I can accommodate the phoshotyrosine moiety for binding, members of Cluster II can not. The conformations of the analyzed side chains in Cluster I are very similar to those in the complex (FIG. 3B) In Cluster II, the Lys203 side chain occupies a large part of the binding pocket, which prevents the fit of the phosphotyrosine.

Analysis of the dihedral angle of Lys203 side chain (Table 1, below) revealed that in the crystal structure of the apo-SH2 domain, the conformation of Lys203 is very similar to the conformation of Cluster II, while the conformation of the side chains in the members of Cluster I are similar to the complex.

TABLE 1
Side chain dihedral angles (in degrees)
of Lys203 in different SH2 structures.

| Structure | x¹ | x² | x³ | x⁴ |
|---|---|---|---|---|
| apo-SH2 | −70.00 | −177.84 | 172.80 | 79.18 |
| complex SH2 | −160.39 | 179.64 | −71.94 | 170.29 |
| Cluster I | −174.91 | −179.88 | 175.61 | 166.54 |
| Cluster II | −66.40 | 162.26 | −143.54 | 75.50 |

The data presented above show that the conformation of the binding pocket of the phoshotyrosine in the crystal structure of apo-SH2 domain and in members of Cluster II is different from the phosphotyrosine—SH2 complex. In these structures the Lys203 occupies some portion of the binding site. Structures in Cluster II, however, had similar side chain conformations in the binding pocket than the SH2-phosphotyrosine.

While the foregoing is a complete description of exemplary embodiments of the invention, it should be evident that various modifications, alternatives and equivalents may be made and used. Accordingly, the above description should not be taken as limiting the scope of the invention which is defined by the metes and bounds of the appended claims.

What is claimed is:

1. A method for selecting a plurality of representative structures of a target molecule, comprising
(i) generating a set of conformational variants of said target molecule,
(ii) forming a plurality of clusters of related conformational variants within said set using a clustering algorithm, and
(iii) selecting a representative structure from each of said plurality of clusters,
wherein representative structures selected from said clusters together constitute said plurality of representative structures.

2. A method of claim 1, wherein said generating includes assembling a set of conformational variants obtained using empirical data.

3. A method of claim 2, wherein said empirical data comprises NMR data.

4. A method of claim 1, wherein said generating includes running a molecular dynamics simulation of said target molecule.

5. A method of claim 4, wherein said target molecule further includes a ligand binding site.

6. A method of claim 5, wherein said molecular dynamics simulation includes modeling a ligand bound at said ligand-binding site.

7. A method of claim 5, wherein said forming comprises using a clustering algorithm that considers only amino acid residues in proximity to said ligand binding site.

8. A method of claim 1, wherein said forming includes using a clustering algorithm based on partitioning around medoids.

9. A method of claim 8, wherein said forming includes using a clustering algorithm comprising fuzzy clustering.

10. A method of claim 1, wherein said forming includes using a clustering algorithm comprising linkage clustering.

11. A method of claim 1, wherein said forming includes using a clustering algorithm comprising hierarchical clustering.

12. A method of claim 1, wherein a representative structure from at least one of said plurality of clusters is a flexible structure.

13. A method of claim 12, wherein a representative structure from at least one of said plurality of clusters is a dynamic pharmacophore developed using conformational variants within said at least one of said plurality of clusters.

14. A method of claim 1, wherein a representative structure from at least one of said plurality of clusters is a rigid structure.

15. A method of claim 14, wherein said representative structure is the same as a structure contained in said at least one of said plurality of clusters.

16. A method of claim 14, wherein said representative structure has the smallest deviation from an average of the structures forming said at least one of said plurality of clusters.

17. A method of claim 1, wherein said target molecule is a representation of a protein.

18. A method of claim 1, wherein said target molecule is based on a crystal structure.

19. A method of claim 1, wherein said target molecule is based on an NMR structure.

20. A method of claim 1, further comprising performing an in silico analysis using said plurality of representative structure and one or more ligands.

21. A method of claim 20, wherein said one or more ligands are small molecules.

22. A method of claim 20, wherein said one or more ligands are peptides.

23. A method of claim 20, wherein said one or more ligands are endogenous ligands.

24. A method of claim 20, wherein said in silico analysis is selected from the group consisting of in silico screening, in silico docking, in silico lead discovery, and in silico lead optimization.

25. A method of claim 24, wherein said in silico analysis comprises screening a plurality of ligands against said molecule.

26. A method of claim 25, wherein said plurality of ligands consists of at least ten ligands.

27. A method of claim 1, wherein said plurality of representative structures consists of at least five representative structures.

28. A method of claim 27, wherein said plurality of representative structures consists of at least ten representative structures.

29. A method of claim 28, wherein said plurality of representative structures consists of at least twenty five representative structures.

30. A method of claim 29, wherein said plurality of representative structures consists of at least fifty representative structures.

31. A method of assessing activity of a plurality of ligands on a target molecule, comprising
providing a plurality of representative structures obtained by clustering a set of conformational variants of said target molecule, and
using said plurality of representative structures as targets in in silico analysis of said plurality of ligands,
wherein results of said in silico analysis are effective to assess activity of said plurality of ligands on said target molecule.

32. A method of claim 31, wherein at least one of said plurality of representative structures is a flexible structure.

33. A method of claim 32, wherein at least one of said plurality of representative structures is a dynamic pharmacophore.

34. A method of claim 31, wherein at least one of said plurality of representative structures is a rigid structure.

35. A method of claim 31, wherein said target molecule is a representation of a protein.

36. A method of claim 31, wherein said plurality of ligands are small molecules.

37. A method of claim 31, wherein said in silico analysis is selected from the group consisting of in silico screening, in silico docking, in silico lead discovery, and in silico lead optimization.

38. A method of claim 31, wherein said plurality of ligands consists of at least ten ligands.

39. A method of claim 31, wherein said plurality of representative structures consists of at least five representative structures.

40. A method of claim 39, wherein said plurality of representative structures consists of at least ten representative structures.

41. A method of claim 40, wherein said plurality of representative structures consists of at least twenty five representative structures.

42. A method of claim 41, wherein said plurality of representative structures consists of at least fifty representative structures.
